(19) United States
(12) Patent Application Publication
(10) Pub. No.: US 2015/0074995 A1
(43) Pub. Date: Mar. 19, 2015

(54) GUIDEWIRE ASSEMBLY METHODS AND APPARATUS

(71) Applicant: Angiometrix Corporation, Bethesda, MD (US)

(72) Inventors: Nitin PATIL, Albany, CA (US); Goncalo SANTOS, Galway (IE); Michael NAGY, Lawrenceville, GA (US)

(73) Assignee: Angiometrix Corporation, Bethesda, MD (US)

(21) Appl. No.: 14/535,165

(22) Filed: Nov. 6, 2014

Related U.S. Application Data
(63) Continuation of application No. PCT/US2013/038147, filed on Apr. 25, 2013.
(60) Provisional application No. 61/644,326, filed on May 8, 2012.

(51) Int. Cl. A61B 5/0215; A61M 25/09

(52) U.S. Cl. CPC A61B 5/0215; A61M 25/09; A61M 2025/09108; A61M 2025/09083; A61M 2025/09183
USPC 29/855; 29/854; 29/825

(57) ABSTRACT

Guidewire assembly methods and apparatus are described for incorporating one or more pressure sensors with one or more electrodes within or along the body of the guidewire. Guidewires may incorporate a number of different sensors within or along the body of the guidewire. To achieve the combination of the pressure sensor and one or more electrodes, various assembly methods and apparatus may be utilized as described in further detail herein.

### CROSS-REFERENCE TO RELATED APPLICATIONS

This application is a continuation of International Application No. PCT/US2013/038147 filed Apr. 25, 2013, which claims the benefit of priority to U.S. Provisional Application No. 61/644,326 filed May 8, 2012, which is incorporated herein by reference in its entirety.

### FIELD OF THE INVENTION

The present invention relates to methods and apparatus for the assembly of guidewires having multiple sensors incorporated within or along the body of the guidewire. In particular, the present invention relates to methods and apparatus for the assembly of guidewires incorporating pressure sensors and one or more electrodes within or along the body of the guidewire.

### BACKGROUND OF THE INVENTION

Guidewires may have a number of sensors or sensor assemblies integrated directly into the guidewire. Such sensor-equipped guidewires may be adapted for measuring various physiological parameters within a patient's body. For instance, sensors typically have one or more cables passed through the guidewire for electrically coupling the sensor element to an electronic assembly.

Guidewires are generally comprised of a hypotube and coiled segment about a core wire which may extend through the length or a partial length of the guidewire. The core wire may be fabricated from stainless steel or Nitinol with the coiled segment fabricated from a wire or braid which provide for flexibility, pushability, and kink resistance to the guidewire. Nitinol wire, used by itself or braided with stainless steel, may further help to increase flexibility and allow the wire to spring back into shape.

Moreover, guidewires have a standard diameter of 0.014 in. and consequently accommodating certain types of sensors or having multiple sensors may be limited by the relatively small space provided by the guidewire. Moreover, guidewires are typically used for insertion into and advancement through the vasculature which can present an extremely tortuous pathway. Thus, the guidewire and any sensors or electrodes along the guidewire may experience relatively large stresses as the guidewire is pushed, pulled, or torqued over a passageway having numerous curves and bends.

Guidewires incorporating one or more electrodes along their length may present additional challenges to guidewire construction and use. For instance, the presence of a plurality of electrodes along the guidewire may require additional conductive wiring passed through the length of the guidewire. Because of the limited space and flexibility required from guidewires, any sensors and/or electrodes positioned along their length are desirably correspondingly constructed.

Consequently, there is a need for guidewire designs which provide for effective construction of a guidewire incorporating one or more electrodes and/or sensors along the length.

### SUMMARY OF THE INVENTION

Guidewires may incorporate a number of different sensors within or along the body of the guidewire. One particular variation may incorporate a pressure sensor with one or more electrodes along the body of the guidewire or at the distal end of the guidewire. A guidewire having one or more electrodes integrated directly along the guidewire body may have a proximal coil attached to an electrode assembly having one or more electrodes and a distal coil attached to a distal end of the electrode assembly. The electrode assembly may further have insulative spacing segments positioned between each of the electrodes to provide for electrical insulation and both the electrodes and spacing segments may be positioned along an electrode assembly or substrate fabricated from insulative polymers, e.g., polyimide. A core wire may extend through the length of the guidewire assembly and may extend partially or entirely through the electrode assembly.

One variation for assembling the guidewire assembly may generally comprise providing a core wire having a tapered distal section, securing a sensor package having one or more conductive wires to the core wire by passing the core wire through a wire receiving channel defined through or along the sensor package, securing the one or more conductive wires to the core wire, and then encasing the one or more conductive wires and the core wire.

Another variation for assembling the guidewire assembly and integrating an electrode assembly may have the proximal end of a truncated core wire and the distal end of core wire or hypotube coupled, joined, or otherwise attached to one another. The electrode assembly may then be advanced over the core wire or hypotube into contact against the proximal end of the distal coil where the electrodes may be electrically coupled to a corresponding conducting wire. The proximal coil may be advanced over the core wire or hypotube into contact against the proximal end of the electrode assembly and the two may be coupled or otherwise attached to one another.

In yet another variation for manufacturing the guidewire, a relatively shortened core wire, e.g., less than 3 cm, may be used or in another variation, a core wire having a length greater than 3 cm, e.g., 20 cm or longer, may be used instead.

In yet another method of attachment to a core wire, the hypotube may have a distal section initially reduced in diameter. The reduced annular portion may then be further processed to remove an arcuate or skived portion which extends from a shoulder of the annular portion down to the distal end of the hypotube such that a tapered distal section is formed. The narrowed end of the distal section may be coupled directly to one another. With the core wire positioned within the distal coil, the electrode assembly may be connected to the proximal end of distal coil while the proximal coil may be connected to the proximal end of the electrode assembly. The various attachments may be achieved through any number of attachment methods, e.g., solder joint, adhesively joined, etc. The attachment may also alternatively use a clip or collar which may be placed over or upon the respective terminal ends.

In yet another variation for manufacturing the guidewire assembly, a core wire may be joined directly to a tapered portion of the hypotube utilizing any number of attachment methods described herein. With the core wire and hypotube coupled, the electrode assembly may be placed along the core wire and the wires passed through the hypotube lumen. The proximal and distal coils may also be attached proximally and distally of the electrode assembly.

Aside from the integration of an electrode assembly along the guidewire, the guidewire assembly may also optionally incorporate one or more sensors along its length. Although any number of sensors for detecting physiological parameters may be integrated, one particular sensor may include a pressure sensor for detecting intravascular fluid pressure. Because of the sensitive nature of the sensor, the pressure sensor diaphragm may be generally insulated from stress, e.g., by omitting coatings or epoxy from areas beneath and/or over the diaphragms. Hence, the regions around the wirebonding connecting the sensor to a substrate or conducting wires are ideal areas for maintaining low stress regions. One example for assembling a pressure sensor having low stress attachment may utilize a platform either formed directly along the core wire or along a separate platform integrated along the core wire or guidewire body used as a floor for attaching the various components of a pressure sensor.

In mounting or attaching the conductive wires along the sensor assembly, various methods may be used for electrically and mechanically bonding the wires along the sensor assembly to maintain a low profile configuration for integrating along the guidewire assembly. One example may be to form a surface mount configuration where an assembly jig may be used. The assembly jig may define a surface having a recess which is sized to receive the substrate or die to be mounted in a secure fitting. One or more channels may be defined along the jig extending from one or more openings directly to the recess. The number of channels may correspond to the number of conductive wires to be surface mounted along the substrate or die. Moreover, the channels may be angled and/or tapered to facilitate guidance of the wires directly to the recess.

The wires may be inserted through a respective opening and placed into proximity to, e.g., a pressure sensor die, positioned within the recess, where the exposed terminal ends may then be soldered or otherwise attached directly to the pressure sensor die. Additionally and/or alternatively, rather than directly attaching the wires to the die surface, an optional endcap fabricated from a metal or plastic may be used to alleviate any stresses which may be imparted between the attachment of wires to the sensor die.

In yet another example for integrating a pressure sensor assembly into a guidewire while maintaining a low profile configuration, the pressure sensor die may be electrically connected directly to one or more conductive wires through attachment via conductive pads utilizing a flip chip type mounting configuration. In the arrangement shown, the one or more conductive wires may be routed through the guidewire and into proximity to the pressure sensor mounting region defined along the guidewire. Within the mounting region, a platform or floor formed along the region may further form recessed region which may be formed as a recess within the platform. With the pressure sensor die inverted relative to the platform, the conductive wires may be electrically connected directly to the respective conductive pads located along the surface of the pressure sensor die. Another example for mounting the pressure sensor die along the guidewire in a low profile may have the pressure sensor die mounted directly to the platform or floor thus allowing for the direct surface mounting of the once or more wires to the respective conductive pads along the surface of the sensor die. This variation also allows for the direct exposure of the diaphragm for sensing physiological parameters. Additionally, this variation may also present the shortest overall height of the pressure sensor relative to the platform thus allowing for a low profile and may also accommodate a relatively wider die.

To electrically couple each of the electrodes and the pressure sensor, multiple conductive wires may be routed through the length of the guidewire but to ensure that the multiple wires are ordered and remain untangled, the wires may be bundled relative to one another. With the conductive wires accordingly stacked and aligned, a first row of wires may be assigned for electrical coupling to the corresponding number of electrodes while the second row of wires may be assigned for electrical coupling to the pressure sensor assembly.

Another example may have the wires processed to have exposed selective regions through the insulative covering at uniform or staggered longitudinal locations for electrically coupling to electrodes or sensors. Alternatively, the terminal ends of the wires may be cut such that the exposed terminal portions are positioned at staggered lengths relative to one another.

In yet another variation for mounting a pressure sensor die having a diaphragm and one or more conductive pads, an electrode assembly may be formed as a composite assembly to which the sensor die may be mounted directly upon. The electrode assembly may be formed to have one or more electrode segments alternated with one or more corresponding insulating segments. Each of the electrode segments may be patterned and removed from a sheet or layer of conductive material such that the electrode segments are individually formed from the sheet or layer or stacked upon one another to form the composite structure.

The electrode assembly may define a core wire receiving channel through the length of the assembly and the outer surfaces of the assembly may define a sensor receiving slot along a length of the assembly as well as an optional slot, e.g., for wiring, etc., along the length of the assembly opposite to the sensor receiving slot. The pressure sensor die may be placed directly within the receiving slot and electrically coupled via respective wirebonds to conductive wires which may be passed through the slot.

Another variation may involve a core wire configured to have a reduced section along its length to provide a sensor mounting section. The reduced section may have a cross-sectional area which is shaped into various configurations to facilitate the mounting or securement of the electrode assembly or other sensors along the section. The conductive segment may define a core wire receiving channel which may be optionally narrowed to provide for a snap fit over the reduced section. Similarly, the insulating segment may also define one or more wire receiving channels as well as a core wire receiving channel. With the desired number of conductive segments formed and the corresponding number of insulating segments also formed, each of the segments may be secured upon the reduced section in an alternating manner as well as secured to one another through various securement methods, e.g., adhesives, mechanical, etc. While the reduced section may be formed to have a cross-sectional area which is shaped into various configurations, the receiving channels defined by the segments may be correspondingly configured as well.

In yet another variation, a discontinuous core wire may be separately attached to the sensor housing. A proximal core wire section and a distal core wire section may each be attached at their respective locations via any number of attachments. Such an arrangement may allow for maintaining adequate space for securement of the sensor along the housing while maintaining a low profile guidewire assembly. Yet another variation may have a portion of the sensor die having the diaphragm extend proximally or distally from the electrode assembly in a cantilevered manner remaining unattached beneath the die. Another variation may incorporate an adjacently secured barrier segment which defines a sensor opening and core wire receiving channel. The sensor opening may be configured as a passage, e.g., rectangular, which is sized to fit the pressure sensor through without necessarily contacting the pressure sensor so as to limit any transfer of stresses.

Yet another variation may be formed from a conductive tube fabricated from a metallic material and attached or otherwise connected over an insulative tube which may provide structural support to the electrode assembly by holding and maintaining a position of each of the conductive segments as well as providing electrical insulation. The insulative tube may define a core wire channel through which the core wire may be positioned. With the conductive tube, portions of the tubing may be removed to provide for space into which the pressure sensor die may be positioned. A structure with similar functional attributes may also be achieved using different manufacturing techniques, e.g., molding the body along with the core wire hole with plastic (non-conductive such as PEEK) and then selectively metalizing the surfaces (e.g., using photo chemical etching) to obtain the conducting pattern to dimensionally align with the conductive pads on the corresponding sensor die.

With the respective channels formed, segments may be formed by the conductive tube by removing selective portions of the material. The formed gaps between each of the conductive segments may have a width to provide for the placement of electrically insulative materials within.

### BRIEF DESCRIPTION OF THE DRAWINGS

FIG. 1 shows a cross-sectional side view of one variation of a guidewire illustrating one or more electrodes positioned along the guidewire body near or at the distal end.

FIG. 2 shows a perspective view of the distal end electrode spacing.

FIG. 3 shows a side view of an electrode assembly having one or more electrodes spaced apart from one another with insulative material positioned between.

FIGS. 4A to 4C illustrate one variation for assembling an electrode assembly along a guidewire.

FIG. 5 shows a cross-sectional side view of another variation for assembling a guidewire having one or more electrodes positioned therealong.

FIG. 6 shows a cross-sectional side view of yet another variation for assembling a guidewire having one or more electrodes.

FIGS. 7A and 7B show side views of a hypotube which may be configured for attachment to a core wire.

FIGS. 7C and 7D show top views of a hypotube attached to a core wire and integrally forming a guidewire having one or more electrodes integrated therealong.

FIG. 7E shows a cross-sectional side view of a core wire to hypotube attachment using a clip or collar for coupling the two portions.

FIGS. 8A to 8D show perspective views of another variation for assembling a guidewire having one or more electrodes positioned therealong.

FIG. 9 shows a detail perspective view for coupling a hypotube to a second tubular member for forming a guidewire.

FIG. 10 shows a partial cross-sectional side view of one method for placing one or more radio-opaque bands on the guidewire.

FIG. 11 shows a partial cross-sectional side view of a guidewire incorporating a continuous core wire through a pressure sensor housing.

FIGS. 12A and 12B show top and side views of a pressure sensor die positioned directly upon a floor of the sensor housing.

FIGS. 13A and 13B illustrate a top view of an assembly jig which may be used to attach one or more conductive wires to a pressure sensor die.

FIGS. 14A and 14B show side and end views of one or more conductive wires and an end cap which may be used to position and maintain the wires relative to a pressure sensor die.

FIGS. 15A to 15D illustrate partial cross-sectional side views of another variation for attaching one or more wires through an endcap and onto a pressure sensor die.

FIGS. 16A to 16C show respective end, side, and top views of a flip-chip assembly method for attaching a pressure sensor die directly to a sensor housing.

FIGS. 17A to 17C show respective end, side, and top views of another method for attaching the pressure sensor die directly to the sensor housing.

FIG. 18 shows a perspective view of a guidewire having the one or more electrodes and a pressure sensor integrated directly into the guidewire.

FIG. 19A shows a cross-sectional end view of one variation for aligning multiple conductive wires through the guidewire.

FIG. 19B shows a cross-sectional end view of another variation of aligned multiple conductive wires having an optional metallization layer coated over the assembly.

FIG. 20A shows one variation for terminating a first set of conductive wires at the one or more electrodes and a second set of conductive wires at the pressure sensor assembly.

FIG. 20B shows a top view of conductive wires which may have offset exposed portions for electrical coupling.

FIG. 20C shows a top view of conductive wires illustrating how the terminal ends may be offset for electrical coupling.

FIG. 21 show a perspective view of a pressure sensor die to be secured to an electrode assembly along a guidewire.

FIGS. 22A and 22B show perspective and end views of another variation of a guidewire having a defined channel for positioning of the pressure sensor.

FIG. 23A shows a side view of a core wire having a reduced section for securing an electrode assembly.

FIGS. 23B and 23C show end views of one variation of conductive and insulative segments for securement to the core wire.

FIGS. 23D and 23E show end views of another variation of conductive segments which may be configured to the core wire having a predetermined cross-sectional shape.

FIG. 24 shows a side view of another variation of a core wire which may be attached as separate portions to a pressure sensor housing.

FIGS. 25A to 25C show side and end views of a pressure sensor die which may be cantilevered to reduce or eliminate any stresses imparted to the sensing diaphragm.

FIGS. 26A to 26C show side, end, and perspective views of another variation of a barrier segment which may be integrated into the guidewire.

FIGS. 27A and 27B show perspective and end views of another variation of a core wire having a tubular pressure sensor housing secured around the core wire.

FIGS. 28A and 28B show end views illustrating an example of how material from the tubular pressure sensor housing may be removed for forming a pressure sensor receiving channel.

FIG. 29 shows a side view of the conductive segments and tubular pressure sensor housing secured upon the core wire.

FIG. 30 shows an end view illustrating a pressure sensor die and conductive wires positioned upon the respective receiving slots.

### DETAILED DESCRIPTION OF THE INVENTION

Guidewires may incorporate a number of different sensors within or along the body of the guidewire. One particular variation may incorporate a pressure sensor with one or more electrodes along the body of the guidewire or at the distal end of the guidewire. To achieve the combination of the pressure sensor and one or more electrodes, various assembly methods and apparatus may be utilized as described in further detail herein.

Examples of guidewires which may incorporate one or more electrodes for assessing various anatomical parameters, such as lumen dimension in vivo, and which may also integrate one or more sensors such as pressure sensors, are shown and described in further detail in the following: U.S. Prov. 61/383,744 filed Sep. 17, 2010; U.S. application Ser. No. 13/159,298 filed Jun. 13, 2011 (U.S. Pub. 2011/0306867); Ser. No. 13/305,610 filed Nov. 28, 2011 (U.S. Pub. 2012/101355); Ser. No. 13/305,674 filed Nov. 28, 20111 (U.S. Pub. 2012/0101369); Ser. No. 13/305,630 filed Nov. 28, 2011 (U.S. Pub. 2012/0071782); Ser. No. 13/709,311 filed Dec. 10, 2012; and Ser. No. 13/764,462 filed Feb. 11, 2013. Each of the applications is incorporated herein by reference in its entirety and is provided for any purpose herein.

Additional examples are also shown and described for the assembly and use of the combination of one or more pressure sensors and one or more electrodes within or along a guidewire in PCT/US2012/034557 filed Apr. 20, 2012 (published as WO 2012/173697 and designating the U.S.) which is also incorporated herein by reference in its entirety for any purpose herein. It is intended that any of these guidewires and other guidewires may utilize any of the methods and apparatus described herein in various combinations as practicable.

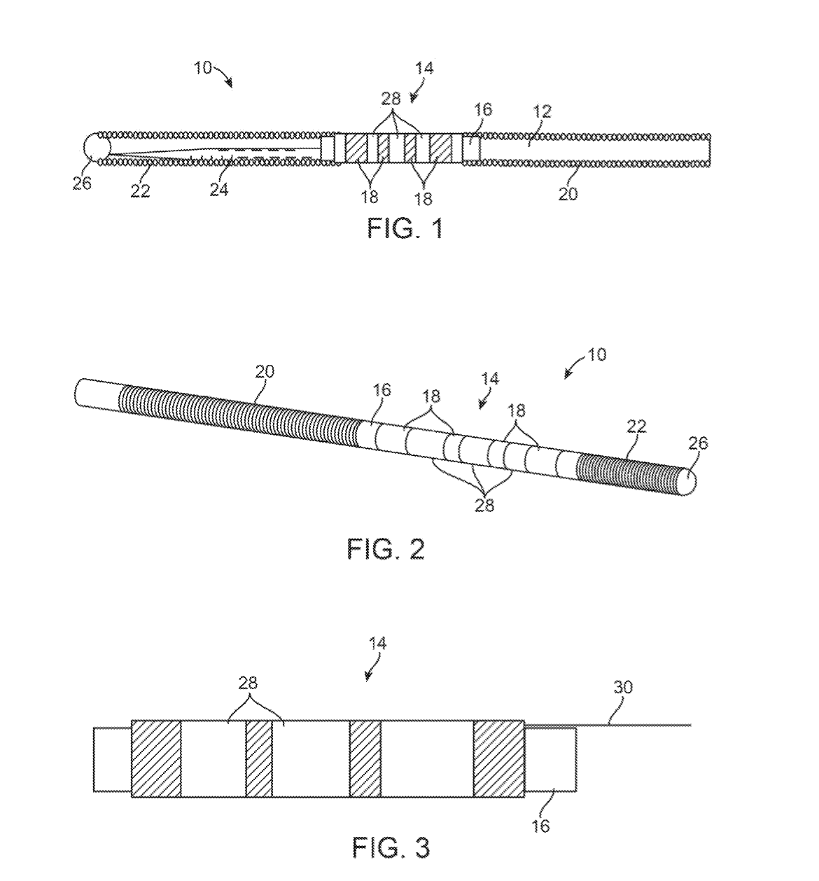

Turning now to FIG. 1, an example of a guidewire 10, e.g., 0.014 in. diameter guidewire, having one or more electrodes integrated directly along the guidewire body is shown in the partial cross-sectional side view. As shown, a hypotube 12, e.g., Nitinol, stainless steel, etc., may have a proximal coil 20. e.g., fabricated from stainless steel, attached to an electrode assembly 14 having one or more electrodes 18 (in this variation four electrodes spaced apart from one another) and a distal coil 22 attached to a distal end of the electrode assembly 14 and terminating in an atraumatic distal tip 26.

The electrode assembly 14 may further have insulative spacing segments 28 positioned between each of the electrodes 18 to provide for electrical insulation and both the electrodes 18 and spacing segments 28 may be positioned along an electrode assembly or substrate 16 fabricated from, e.g., polyimide. One or both of the proximal coil 20 and/or distal coil 22 may be fabricated from a variety of biocompatible materials which also provide sufficient structural strength, e.g., platinum (Pt), platinum-iridium alloys (Pt/Ir), etc. A core wire 24 may extend through the length of the guidewire assembly 10 and may extend partially or entirely through the electrode assembly 14. The core wire 24 may be fabricated from, e.g., stainless steel, Nitinol, etc., and may also be tapered into a relatively smaller diameter the further distal the core wire 24 extends.

Another view of the guidewire assembly 10 is shown in the perspective view of FIG. 2, which illustrates the spacing of the electrodes 18 with the adjacent insulative spacing segments 28 between each of the electrodes 18. Also shown are the proximal and distal coils 20, 22, respectively, and the smooth outer surface presented by the assembly 10. FIG. 3 shows a side view of the electrode assembly 16 removed from the guidewire body to illustrate the positioning of the electrodes 18 relative to the spacing segments 28 and how the one or more conducting wires 30 electrically coupled to each of the respective electrodes 18 may extend proximally from the assembly 16.

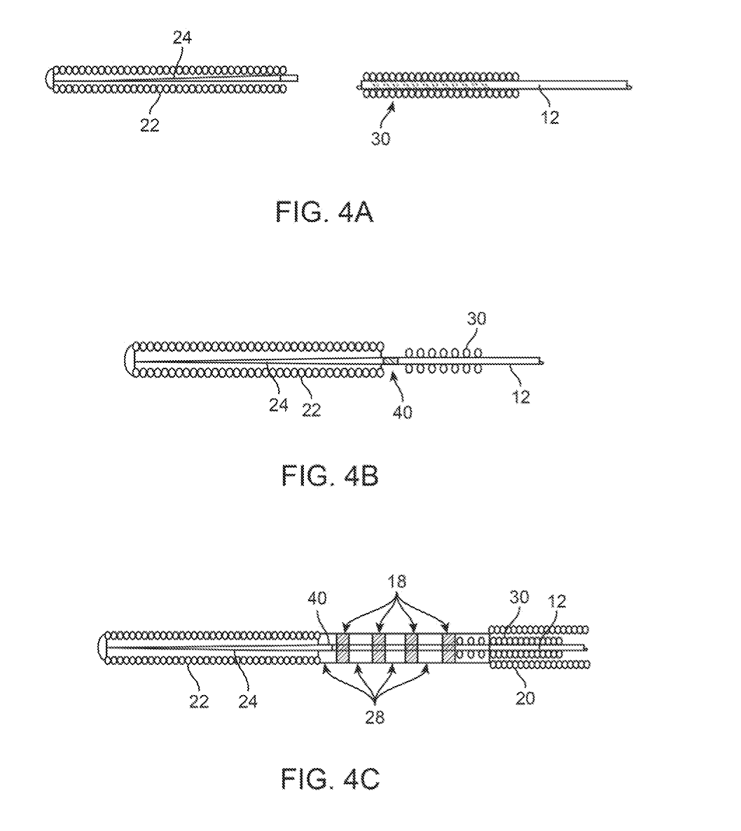

FIGS. 4A to 4C illustrate one variation for assembling the guidewire assembly 10 and integrating an electrode assembly 16. As shown in FIG. 4A, core wire 24 may be secured within a portion of the distal coil 22 where the core wire 24 having an outer diameter of, e.g., 0.005 in., may be tapered to an outer diameter of, e.g., 0.002 in., over a length of, e.g., 3 cm. A core wire or hypotube 12 separate from the core wire 24 may have one or more conducting wires 30 for attachment to the electrodes twisted, spooled, or otherwise wrapped around the core wire or hypotube 12. With this assembly, the proximal end of the core wire 24 and the distal end of core wire or hypotube 12 may be coupled, joined, or otherwise attached at an attachment 40, e.g., laser welded joint, to one another, as shown in the side view of FIG. 48. In this embodiment two different core wires are described since the materials of the core wires can be different (e.g. Nitinol for the distal core wire and stainless steel for the proximal core wire) to take advantage of the material properties and satisfy different performance requirements of the wire (e.g., high kink resistance offered by a Nitinol distal core versus high stiffness along the proximal shaft which may be derived using a stainless steel core). However, it should be noted that a single continuous core wire material (e.g., stainless steel) may be used for the wire construction.

The electrode assembly having the electrodes 18 and insulative spacing segments 28 may then be advanced over the core wire or hypotube 12 and conducting wires 30 into contact against the proximal end of the distal coil 22 where the electrodes 18 may be electrically coupled to a corresponding conducting wire 30. The proximal coil 20 may be advanced over the core wire or hypotube 12 into contact against the proximal end of the electrode assembly and the two may be coupled or otherwise attached to one another, as shown in the side view of FIG. 4C. It should be noted that in place of a coil 20 a suitable polymer (e.g., polyimide or nylon) can be used to encapsulate the core and the conducting wires through the length of the guidewire.

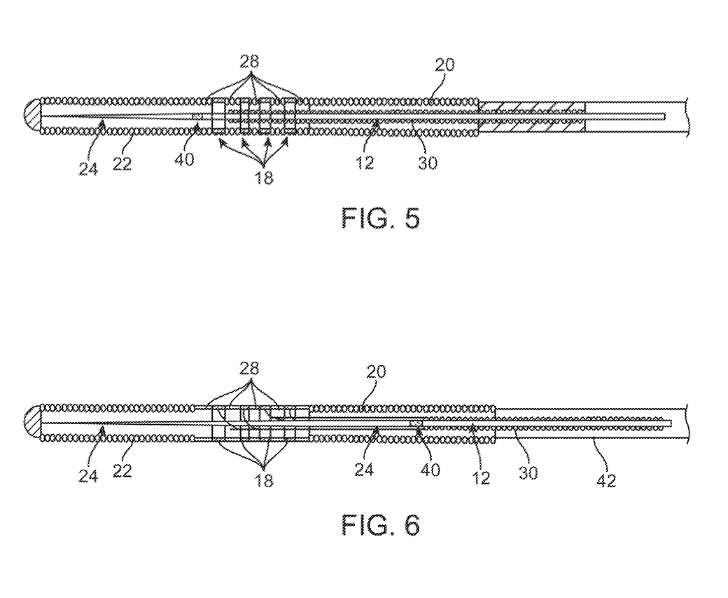

In yet another variation for manufacturing the guidewire, FIG. 5 shows a partial cross-sectional side view of a guidewire assembly having a relatively shortened core wire 24, e.g., less than 3 cm, such that the proximal end of the core wire 24 is positioned within the distal coil 22. The distal end of core wire or hypotube 12 is correspondingly longer and may extend distally through the electrode assembly and at least partially into and through the proximal end of the distal coil 22. The addition of a hypotube 42, e.g., laser cut, may be attached or coupled to a proximal end of the proximal coil 20.

FIG. 6 shows yet another variation where the core wire 24 may be relatively lengthened such that the core wire 24 has a length greater than 3 cm, e.g., 20 cm or longer, and may extend proximally such that the terminal end is positioned proximally of the electrode assembly and within the proximal coil 20. The attachment 40 between the proximal end of the lengthened core wire 24 and the distal end of the core wire or hypotube 12 may be accordingly positioned proximal to the electrode assembly and within the proximal coil 20 or within the hypotube 42.

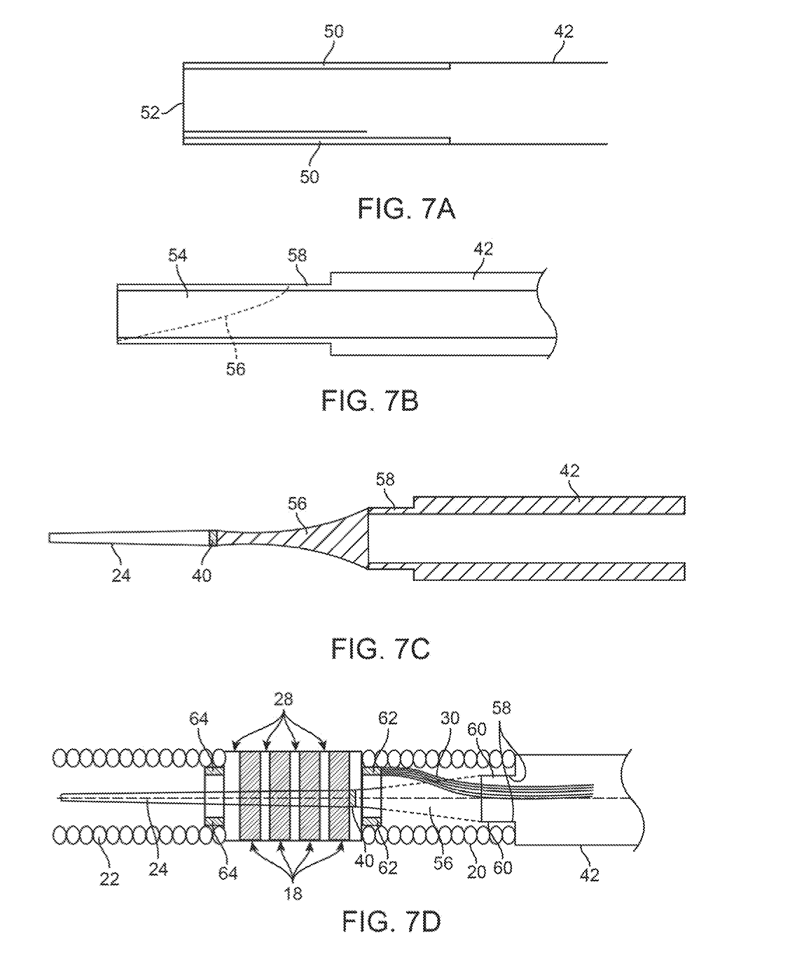

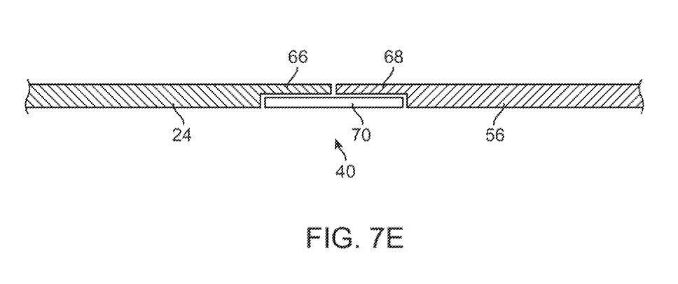

FIGS. 7A to 7D illustrate yet another method of attachment to a core wire 24 through an electrode assembly and directly to a hypotube 42. In this variation, the hypotube 42 may have a distal section initially reduced in diameter from an outer diameter of, e.g., 0.014 in., down to an outer diameter of, e.g., 0.012 in., along a length of less than, e.g., 1.0 in., as shown by the reduced annular portion 50 in the side view of FIG. 7A. The reduced annular portion 50 may then be further processed to remove an arcuate or skived portion 54 which extends from a shoulder 58 of the annular portion 50 (e.g., forming a length of 0.315 in.) down to the distal end 52 of the hypotube 42 such that a tapered distal section 56 is formed, as shown in the side view of FIG. 7B.

As seen in the top view of FIG. 7C, the resulting tapered distal section 56 may be narrowed down to a width of, e.g., 0.005 in., which may correspond to a diameter of the core wire 24. The narrowed end of the distal section 56 may be coupled directly to one another via attachment 40 (using any of the attachment methods herein) such that the core wire 24 and connected distal section 56 form a direct and integrated structure. With core wire 24 positioned within the distal coil 22, the electrode assembly may be connected to the proximal end of distal coil 22 via attachment 64 while proximal coil 20 may be connected to the proximal end of the electrode assembly via attachment 62 and to the shoulder 58 of hypotube 42 via attachment 60, as shown in the partial cross-sectional side view of FIG. 7D. The various attachments may be achieved through any number of attachment methods, e.g., solder joint, adhesively joined, etc.

While the attachment 40 between the core wire 24 and the tapered distal section 56 may be achieved via any of the attachment methods described above, the attachment may also alternatively use a clip or collar 70 (e.g., platinum tube, etc.) which may be placed over or upon the respective terminal ends. The terminal end of the core wire 24 may alternatively define a reduced section 66 (e.g., having a diameter of 0.012 in.) while the terminal end of the distal section 56 may similarly define a reduced section 68 (also having a similarly reduced diameter of 0.012 in.). The clip or collar 70 may be placed over each of the reduced sections 66, 68 and crimped or attached accordingly, e.g., laser or spot welded to respective reduced sections 66, 68, as shown in the detail side view of FIG. 7E.

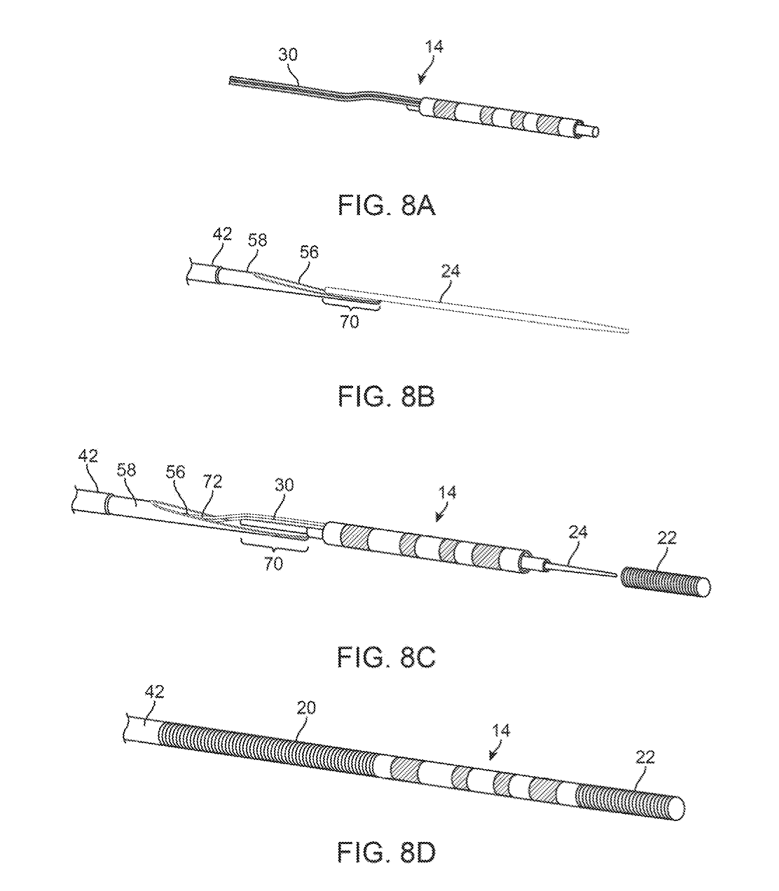

In yet another variation for manufacturing the guidewire assembly, FIGS. 8A to 8D show perspective views illustrating another example of how an electrode assembly 14 having one or more corresponding conductive wires 30, as shown in FIG. 8A, may be assembled with a core wire 24 joined directly to a tapered portion 56 of the hypotube 42, as shown in FIG. 8B. A proximal section of the core wire 24 may be joined along an attachment region 70 to a distal section 56 of the tapered hypotube 42. The core wire 24 may be attached utilizing any number of attachment methods described herein. With the core wire 24 and hypotube 42 coupled, the electrode assembly 14 may be placed along the core wire 24 and the wires 30 passed through the hypotube lumen 72, as shown in FIG. 8C. The proximal and distal coils 20, 22 may also be attached proximally and distally of the electrode assembly 14, as shown in FIG. 8D and as described herein.

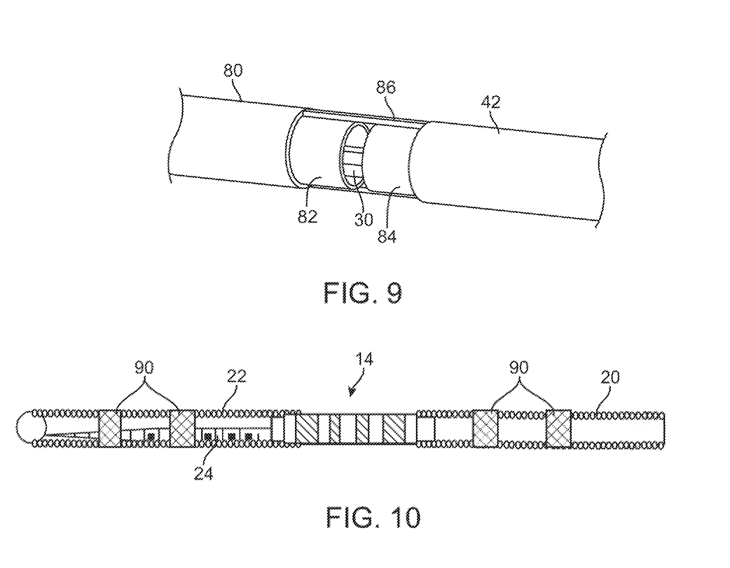

Additionally and/or optionally, in the event that a second hypotube 80 is joined to the hypotube 42, a reduced section 82 of second hypotube 80 and a reduced section 84 of hypotube 42 may be coupled to one another via a clip or collar 86, e.g., platinum tube, which may be laser or spot welded to the respective reduced sections 82, 84, as shown in the detail perspective view of FIG. 9.

In the event that any of the guidewire assemblies described herein require one or more radio-opaque markers to be integrated along its length, any number of crimping or attachment methods may be utilized. One additional and/or optional variation is shown in the partial cross-sectional side view of FIG. 10 which shows a guidewire assembly having one or more radio-opaque markers 90 attached. Such markers 90 may be attached, e.g., by gold solder formed upon the respective coiled sections. By omitting any metal components for the markers 90, the number of steps may be reduced in manufacturing the guidewire and may further avoid any increase in guidewire profile.

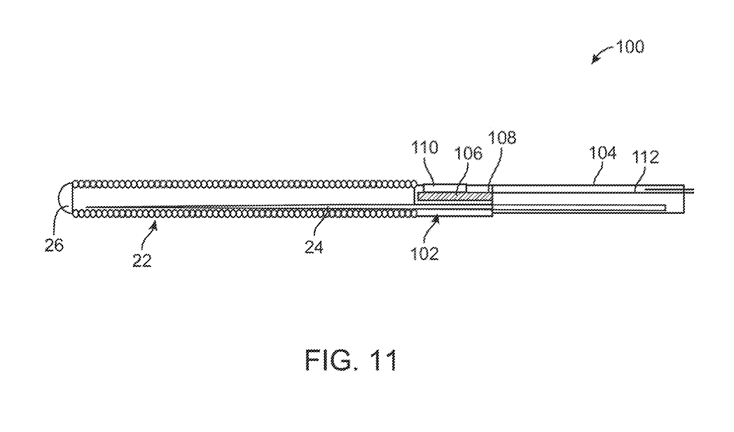

Aside from the integration of an electrode assembly along the guidewire, the guidewire assembly may also optionally incorporate one or more sensors along its length. Although any number of sensors for detecting physiological parameters may be integrated, one particular sensor may include a pressure sensor for detecting intravascular fluid pressure. A partial cross-sectional side view is shown in FIG. 11 to illustrate an example of the relative positioning of the pressure sensor within or along the guidewire. As shown, the pressure sensing guidewire assembly 100 may have the pressure sensor housing 102 secured along the guidewire body at or near the terminal end 26 of the guidewire such that the diaphragm 106 of substrate 108 is exposed through slot 110 for contact with the surrounding fluid. The guidewire assembly 100 may further include the core wire 24 passing through the guidewire and sensor housing 102. The distal coiled body 22 of the guidewire assembly 100 may extend distally from the sensor housing 102 while the leads 112 connecting the diaphragm 106 and substrate 108 may pass proximally through the guidewire body 104 that is encapsulated in one or more polymers along its length may also be seen for connection to another module, e.g., a processor, monitor, etc., located outside the patient's body in use.

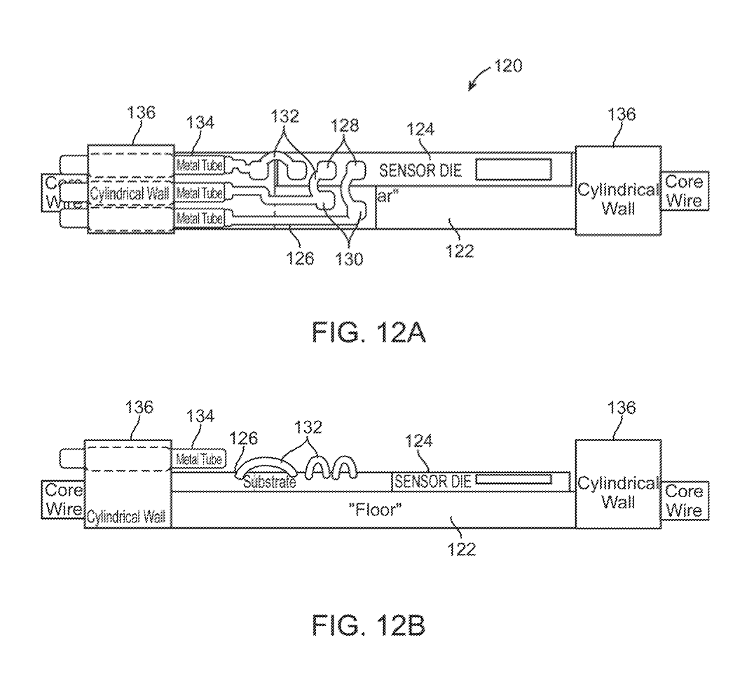

Because of the sensitive nature of the sensor, the pressure sensor diaphragm may be generally insulated from stress, e.g., by omitting coatings or epoxy from areas beneath and/or over the diaphragms. Hence, the regions around the wirebonding connecting the sensor to a substrate or conducting wires are ideal areas for maintaining low stress regions. One example for assembling a pressure sensor having low stress attachment may be seen in the top and side views of FIGS. 12A and 12B which show pressure sensor assembly 120 which may be integrated along the guidewire assembly. As shown in FIG. 12A, a platform 122 either formed directly along the core wire or along a separate platform integrated along the core wire or guidewire body may be used as a floor for attaching the various components of a pressure sensor. The platform 122 may be secured between apposed cylindrical walls 136 and the walls 136 and platform 122 may be secured to the core wire or a distal and proximal portion of a core wire may be attached at respective distal and proximal locations along the cylindrical walls 136.

As shown, the pressure sensor die 124 and substrate 126 (e.g., PCB substrate, flex circuit, etc.) may be attached directly to the floor 122 between the walls 136. One or more conductive wires 134 may be secured through the proximal cylindrical wall 136 such that the exposed terminal ends of the wires 134 may be electrically attached to the substrate 126. Electrical connections between the pressure sensor die 124 and substrate 126 may be made by wirebonds 132 coupling respective conductive pads 128, 130 which are also electrically coupled to the one or more conductive wires 134. The wirebonds 132 may have a loop height generally about, e.g., 0.001 to 0.002 in., above the surface of the substrate 126 with a wirebond outer diameter of about, e.g., 0.001 in., as shown in the side view of FIG. 12B. With this configuration of the pressure sensor die 124 and substrate 126 placed directly upon the floor 122, the assembly may maintain a low profile for integration along the guidewire body. Aside from utilizing wirebonds, flip chip methods of bonding using stud bumps can also be utilized to save space (as described in further detail herein).

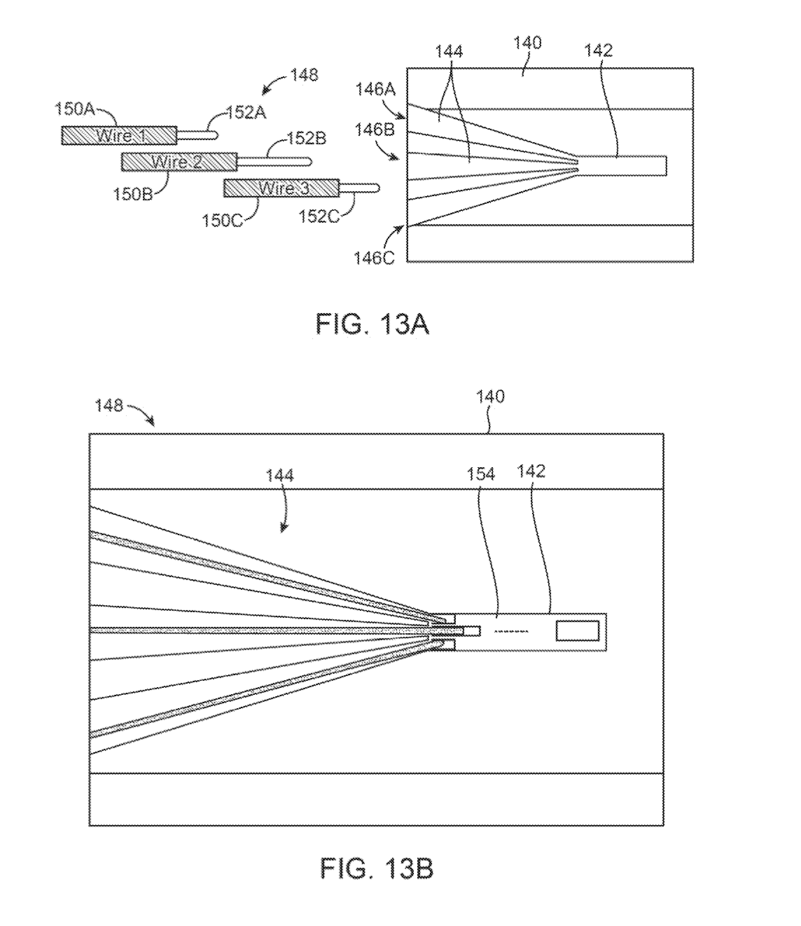

In mounting or attaching the conductive wires along the sensor assembly, such as the substrate 126 or pressure sensor die 124, various methods may be used for electrically and mechanically bonding the wires along the sensor assembly to maintain a low profile configuration for integrating along the guidewire assembly. One example may be to form a surface mount configuration where an assembly jig 140 such as the one shown in the top view of FIG. 13A may be used. The assembly jig 140 may define a surface having a recess 142 which is sized to receive the substrate or die to be mounted in a secure fitting. One or more channels 144 may be defined along the jig 140 extending from one or more openings 146A, 146B, 146C directly to the recess 142. The number of channels 144 may correspond to the number of conductive wires 148 to be surface mounted along the substrate or die. Moreover, the channels 144 may be angled and/or tapered to facilitate guidance of the wires 148 directly to the recess 142.

The conductive wires 150A, 150B, 150C, shown in this example as three wires although fewer or greater number of wires may be used, may each have their terminal ends 152A, 152B, 152C exposed for attachment, as shown in FIG. 13A. The wires 150A, 150B, 150C may be inserted through a respective opening 146A, 146B. 146C and placed into proximity to, e.g., a pressure sensor die 154, positioned within the recess 142, where the exposed terminal ends 152A, 152B, 152C may then be soldered or otherwise attached directly to the pressure sensor die 154, in this example although other substrates may also be used, and as shown in FIG. 13B.

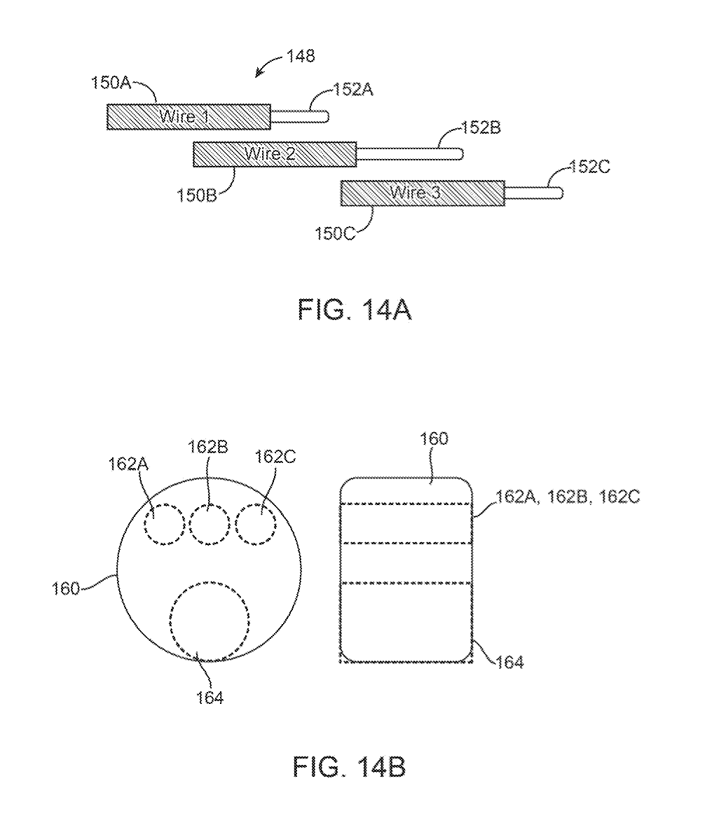

Additionally and/or alternatively, rather than directly attaching the wires 148 to the die surface, an optional endcap 160 fabricated from a metal or plastic may be used to alleviate any stresses which may be imparted between the attachment of wires 148 to the sensor die 154. An example is shown in the end and side views of FIGS. 14A and 14B which illustrate a cylindrical endcap 160 (also shown as the cylindrical wall 136 in FIGS. 12A and 12B). The endcap 160 may have a diameter consistent with the diameter of the guidewire and may further define one or more wire receiving openings 162A, 162B, 162C each having a diameter of, e.g., 0.0015 to 0.003 in, for receiving a corresponding wire. Fewer than three or more than three openings may be utilized depending upon the number of wires used. Alternatively, the openings may be sized to accommodate two or more wires and the openings may be sized in different configurations depending upon the number of wires passed through the openings. An additional core wire opening 164 having a diameter of, e.g., 0.003 to 0.006 in., may also be defined through the endcap 160. The position of the core wire opening 164 can either be concentric or off-centered depending on space availability and performance requirements.

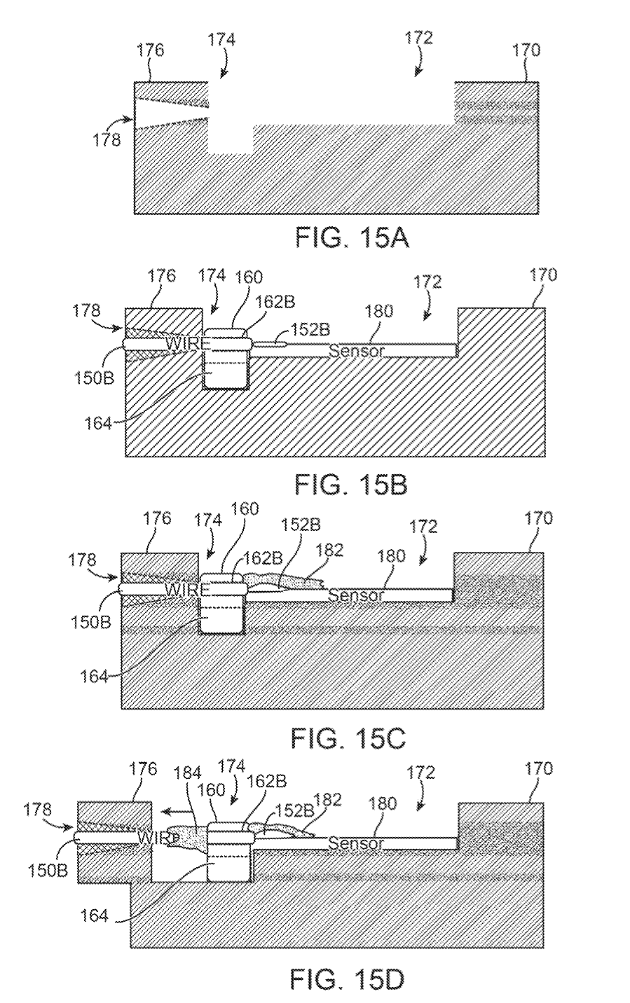

FIGS. 15A to 15D show partial cross-sectional side views of another variation for surface mounting or attaching conductive wires to a substrate or pressure sensor die using the endcap 160. As shown in FIG. 15A, the assembly jig 170 may similarly define a recess 172 sized to receive a substrate or sensor die upon which the wires are to be connected. The jig 170 may further define an endcap channel or recess 174 at a location adjacent to where the wire channels 178 are defined through a wire guide 176. The endcap channel or recess 174 may extend into the jig 170 at a depth sufficient to accommodate the diameter of the endcap 160 such that the openings 162B through the endcap 160 align with the wire channels 178 and substrate or die when positioned within the recess 172, as shown in the partial cross-sectional side view of FIG. 15B.

With the one or more wires 150B inserted through the corresponding wire channel 178 and endcap opening 162B, the exposed terminal end 152B may be placed upon the conductive pad along the pressure sensor die 180 positioned adjacent to the endcap 160 and within the recess 172. The terminal end 152B may then be attached or appropriately surface-mounted upon the sensor 180 through any number of attachment methods such as solder, conductive epoxy, etc. optionally followed by an additional overcoat 182, as shown in FIG. 15C. The wire guide 176 may be slidably attached to the remainder of the jig 170 such that the guide 176 may be retracted to expose the endcap 160. The junction formed between the entry location of the wire 150B and endcap 160 may also be attached relative to one another using any number of attachment methods described above. The attachment may be followed by an optional overcoat 184, as shown in FIG. 15D. Once the attachment has been completed, the sensor 180, endcap 160, and wire 152B assembly may be removed from the jig 170 for assembly into the guidewire.

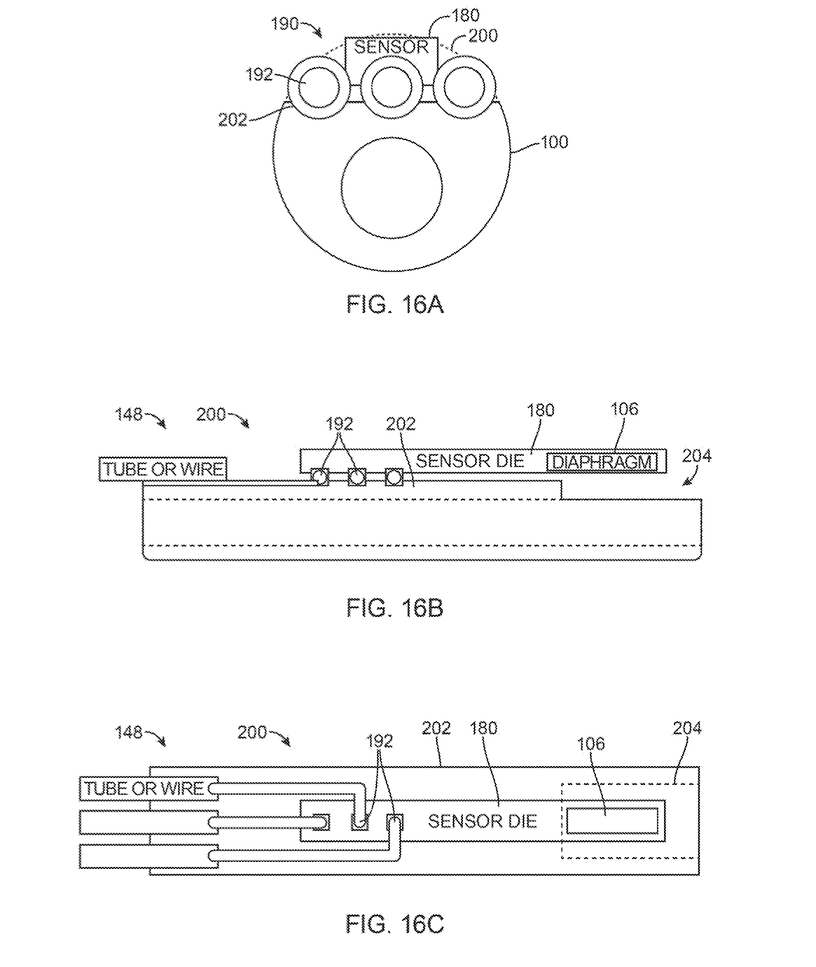

In yet another example for integrating a pressure sensor assembly 190 into a guidewire while maintaining a low profile configuration, FIGS. 16A to 16C show another variation in the end, side, and top views where the pressure sensor die 180 may be electrically connected directly to one or more conductive wires 148 through attachment via conductive pads 192 utilizing a flip chip type mounting configuration. In the arrangement shown, the one or more conductive wires 148 may be muted through the guidewire and into proximity to the pressure sensor mounting region 200 defined along the guidewire. Within the mounting region 200, a platform or floor 202 formed along the region may be further form recessed region 204 which may be formed as a recess within the platform 202. With the pressure sensor die 180 inverted relative to the platform 202, the conductive wires 148 may be electrically connected directly to the respective conductive pads 192 located along the surface of the pressure sensor die 180. Moreover, by inverting the pressure sensor die 180 the location of the diaphragm 106 may also be inverted to become placed in apposition to the platform 202, as shown in the side view of FIG. 16B, directly over the recessed region 204, as further shown in the top view of FIG. 16C. Hence, the diaphragm 106 may remain exposed over the region 204 and uninhibited so as to allow for the sensing of physiological parameters such as fluid pressure. It is also possible to make the diaphragm 106 and the conductive pads on the sensor die 192 on the opposite surfaces of the pressure sensor by a technique referred to as Through Silicone Via (TSV). In such a case, the same technique of using the flip chip method described above can be utilized with or without having any recess in the platform 202.

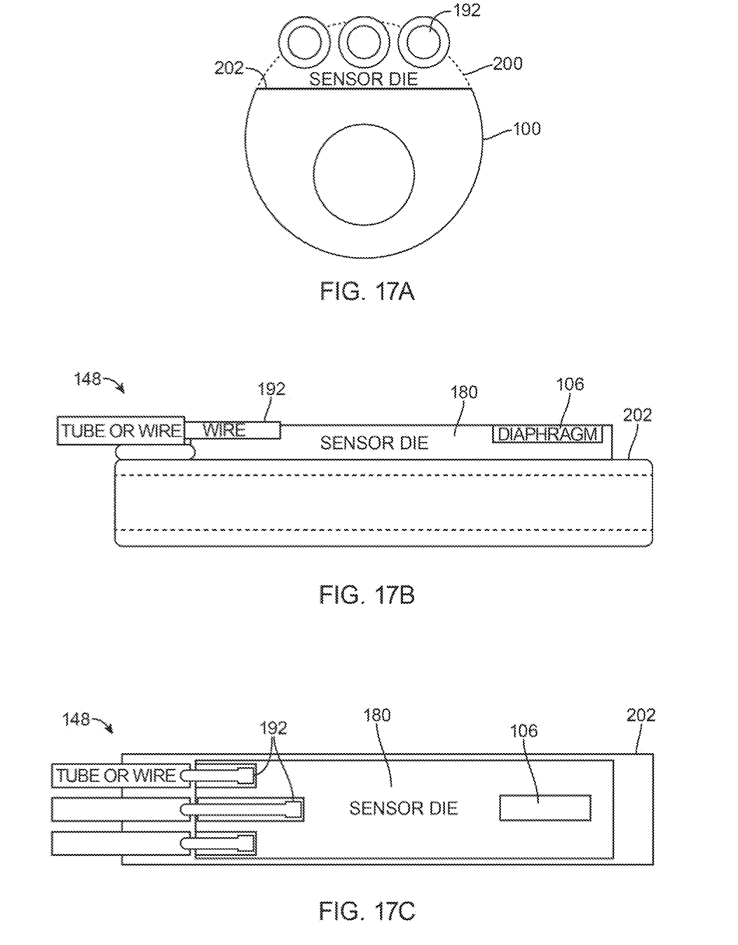

Another example for mounting the pressure sensor die 180 along the guidewire in a low profile is further shown in the end, side, and top views of FIGS. 17A to 17C. In this variation, the pressure sensor die 180 may be mounted directly to the platform or floor 202 thus allowing for the direct surface mounting of the once or more wires 148 to the respective conductive pads 192 along the surface of the sensor die 180. This variation also allows for the direct exposure of the diaphragm 106 for sensing physiological parameters. Additionally, this variation may also present the shortest overall height of the pressure sensor relative to the platform 202 thus allowing for a low profile and may also accommodate a relatively wider die.

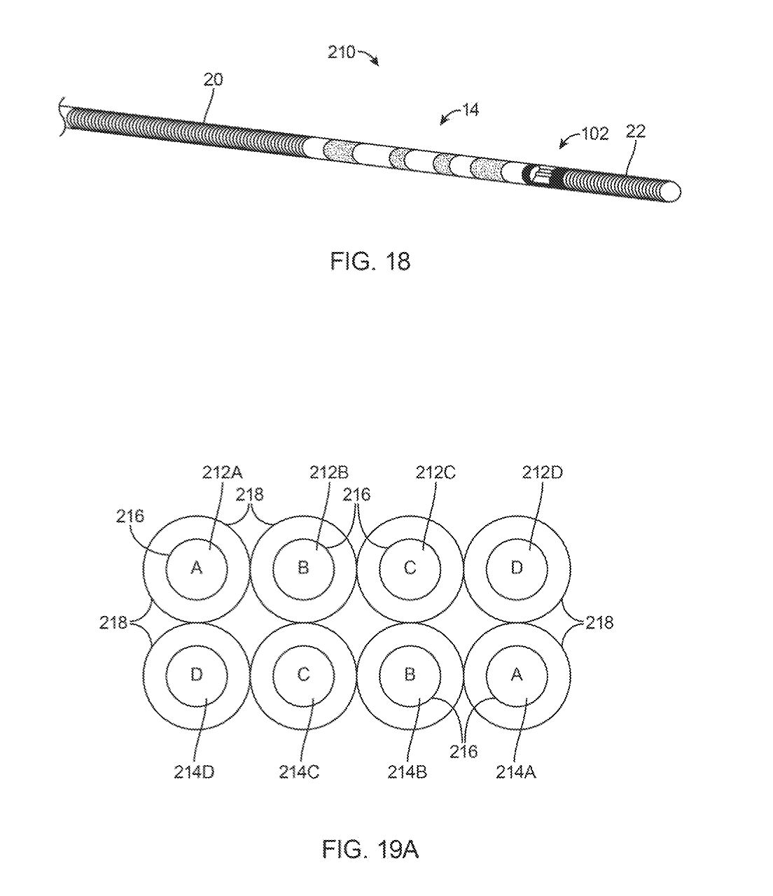

FIG. 18 illustrates a perspective view of an electrode and pressure sensing assembly integrated along a single guidewire 210. Although the electrode assembly 14 is shown proximal to the pressure sensing housing 102 along the guidewire body, the pressure sensing housing 102 may alternatively be located proximal to the electrode assembly 14 instead. To electrically couple each of the electrodes and the pressure sensor, multiple conductive wires may be routed through the length of the guidewire but to ensure that the multiple wires are ordered and remain untangled, the wires may be bundled relative to one another.

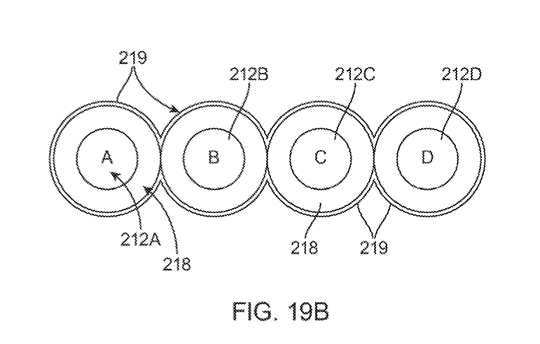

FIG. 19A shows a cross-sectional end view illustrating how multiple conductive wires 212A, 212B, 212C, 212D and conductive wires 214A. 214B, 214C, 214D may be positioned relative to one another. While shown in this example with eight wires, this is intended to be illustrative and fewer than eight or greater than eight wires may be utilized in practice. Nonetheless, each of the wires may have a base coating 216, e.g., polyimide, and a further polymer matrix 218, e.g., pellathane matrix, surrounding each of the wires and forming an attachment to adjacent wires such that the wires form an ordered and stacked ribbon. Another variation to the conductor configuration may include an additional layer of metallization 219 over the coated polymer matrix 218, as shown in the end view of FIG. 19B. Such a metallization layer 219 may have a thickness of, e.g., 2 to 5 microns, and can be added by processes well known in the art such as chemical vapor deposition where metals such as copper, gold, aluminum, etc., are commonly deposited on a substrate (such as polyimide or other polymers). In this case, the metallization layer 219 may be deposited over the polymer matrix 218. The metallization layer 219 can serve several functions such as electrically isolating the conducting wires from Electro Magnetic (EM) Coupling thus providing an EM shield. This may be desirable in many sensor applications where external noise coupling needs to be avoided.

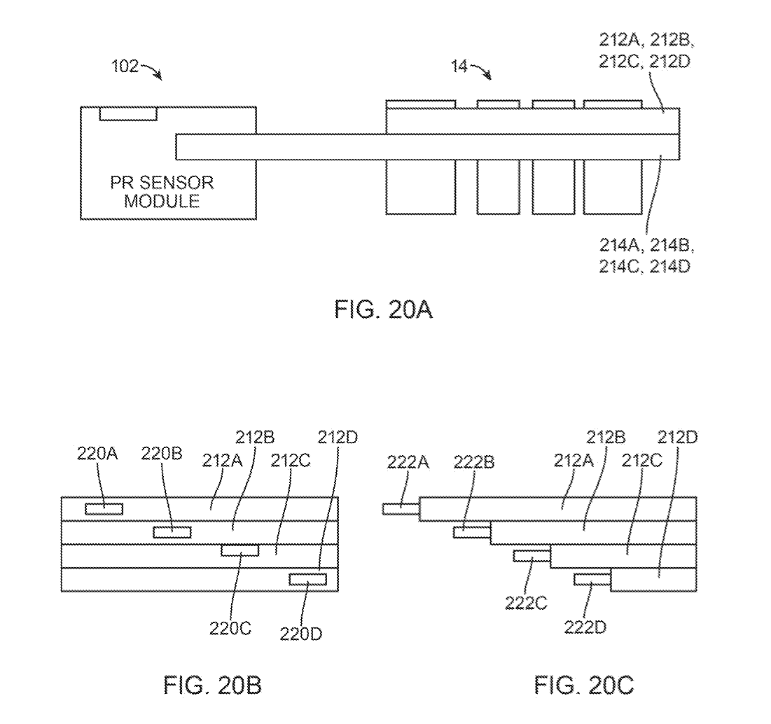

With the conductive wires accordingly stacked and aligned, a first row of wires, e.g., wires 212A, 212B, 212C, 212D, may be assigned for electrical coupling to the corresponding number of electrodes while the second row of wires, e.g., wires 214A, 214B, 214C, 214D, may be assigned for electrical coupling to the pressure sensor assembly 102. FIG. 20A shows an example of how the first row of wires may terminate at the electrode assembly 14 through the guidewire while the second row of wires may continue on through the guidewire for coupling to the pressure sensor assembly 102.

Another example is illustrated in the top view of FIG. 20B which shows how portions of the conductive wires may be processed to have exposed selective regions 220A, 220B, 220C, 220D through the insulative covering at uniform or staggered longitudinal locations for electrically coupling to electrodes or sensors. Alternatively, the terminal ends of the wires may be cut such that the exposed terminal portions 222A, 222B, 222C, 222D are positioned at staggered lengths relative to one another, as shown in the top view of FIG. 20C.

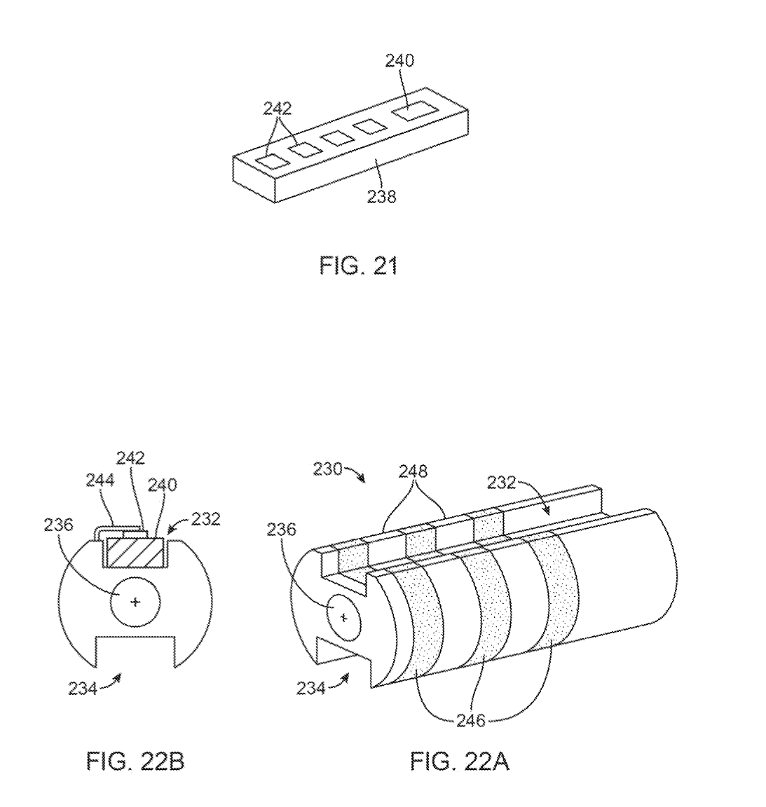

In yet another variation for mounting a pressure sensor die 238 having a diaphragm 240 and one or more conductive pads 242, as shown in the perspective view of FIG. 21, FIG. 22A shows a perspective view of an electrode assembly 230 which may be formed as a composite assembly to which the sensor die 238 may be mounted directly upon. The electrode assembly 230 may be formed to have one or more electrode segments 246 (e.g., fabricated from a conductive material such as gold or other metallic) alternated with one or more corresponding insulating segments 248 (e.g., fabricated from polyimide or other polymeric material or another electrically insulative material). Each of the electrode segments 246 may be patterned and removed (e.g., EDM, laser cut, etc.) from a sheet or layer of conductive material such that the electrode segments 246 are individually formed from the sheet or layer or stacked upon one another to form the composite structure.

The electrode assembly 230 may define a core wire receiving channel 236 through the length of the assembly and the outer surfaces of the assembly may define a sensor receiving slot 232 along a length of the assembly as well as an optional slot 234, e.g., for wiring, etc., along the length of the assembly opposite to the sensor receiving slot 232. The pressure sensor die 238 may be placed directly within the receiving slot 232 and electrically coupled via respective wirebonds 244 to conductive wires which may be passed through slot 234, as shown in the partial cross-sectional end view of FIG. 22B. Once the sensor die 238 has been wirebonded, the assembly may be potted using an appropriate material to provide for further mechanical strength and structural stability. The potting may be restricted to the conductive pads while remaining free from the sensor diaphragm 240. While wirebonding is shown as the attachment method from the sensor conductive pads to the conducting elements 246, other methods such as flip chip as described above can be utilized to attach the sensor die directly on the base of the channel 232. In this case the sensor dies may be fabricated such that the conducting pads 242 and the diaphragm 240 are on opposite faces of the sensor die 238. This can be achieved by sensor die fabrication methods know in the art such as TSV. Using such a method may yield a desirable profile to package the sensor along a 0.014 in. guidewire.

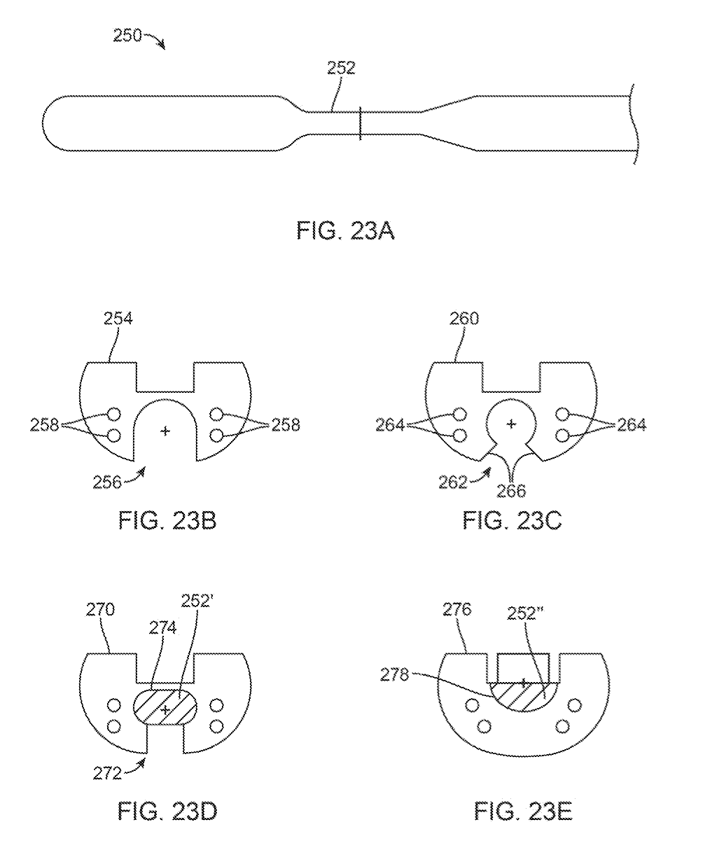

FIG. 23A illustrates a side view of a core wire 250 which may be configured to have a reduced section 252 along its length to provide a sensor mounting section. The reduced section 252 may have a cross-sectional area which is shaped into various configurations to facilitate the mounting or securement of the electrode assembly or other sensors along the section. One variation is illustrated in the end view of FIG. 23B which illustrates a conductive segment 254 and FIG. 23C which illustrates an insulating segment 260 which may be attached to the core wire 250 adjacent to the conductive segment 254. The conductive segment 254 may be formed to have one or more wire receiving channels 258 for passage of the conducting wires and the segment 254 may further define a core wire receiving channel 256 which may be optionally narrowed to provide for a snap fit over the reduced section 252. Similarly, the insulating segment 260 may also define one or more wire receiving channels 264 as well as a core wire receiving channel 262. The receiving channel 262 defined by the segment 260 may further define narrowed receiving members 266 which allow for the segment 260 to be snapped into place upon the reduced section 252. With the desired number of conductive segments 254 formed and the corresponding number of insulating segments 260 also formed, each of the segments 254, 260 may be secured upon the reduced section 252 in an alternating manner as well as secured to one another through various securement methods, e.g., adhesives, mechanical, etc.

While the reduced section 252 may be formed to have a cross-sectional area which is shaped into various configurations, the receiving channels defined by the segments may be correspondingly configured as well. An example is shown in the end view of FIG. 23D which illustrates a conductive segment 270 defining a core wire receiving channel 272 which is formed into a receiving section 274 correspondingly shaped for placement upon a keyed core wire section 252′, e.g., elliptical, rectangular, etc. Another variation is shown in the end view of FIG. 23E which also shows a conductive segment 276 having a configured receiving section 278 for securement to a correspondingly keyed core wire section 252″, e.g., semi-spherical, etc. In this variation, the pressure sensor die may also be placed directly upon the reduced section 242″. Other configurations of the reduced section 252 as well as the corresponding shapes defined by the segments may be utilized in other variations.

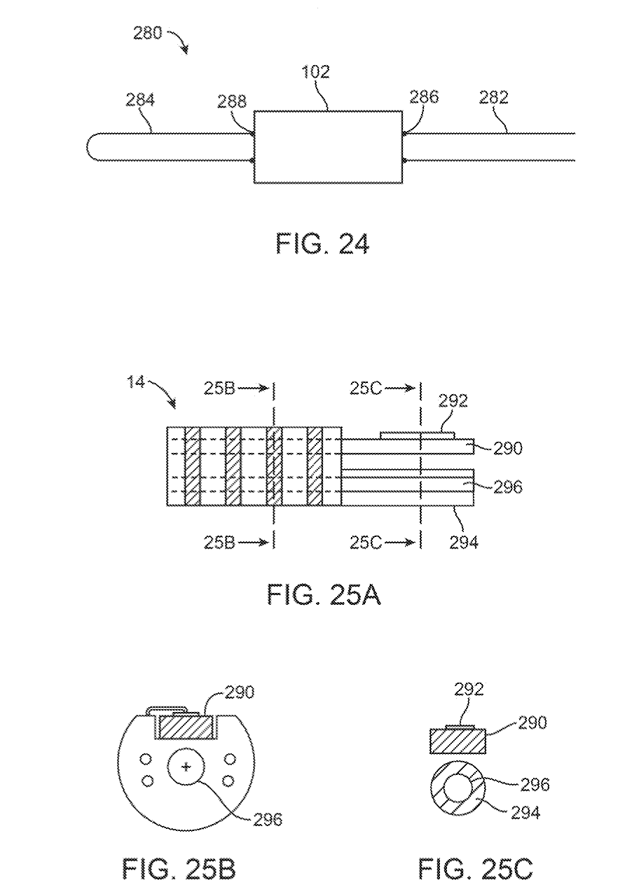

In yet another variation, FIG. 24 shows a side view of an assembly having a discontinuous core wire 280 which may be separately attached to the sensor housing 102. A proximal core wire section 282 and a distal core wire section 284 may each be attached at their respective locations via any number of attachments 286, 288. e.g., welded joint, adhered attachment, etc. Such an arrangement may allow for maintaining adequate space for securement of the sensor along the housing 102 while maintaining a low profile guidewire assembly.

FIG. 25A shows a side view of yet another variation where the exposed diaphragm 292 of the pressure sensor die 290 may be isolated from any stresses which may be imparted by the guidewire or sensor die. The pressure sensor die 290 may be attached through the electrode assembly 14 such that the portion of the die 290 having the diaphragm 292 may extend proximally or distally from the electrode assembly 14 in a cantilevered manner remaining unattached beneath the die. A polymeric housing 294 defining a core wire receiving channel 296 may also extend through the electrode assembly 14 adjacent to the cantilevered sensor die 290, as shown in the end views of FIGS. 25B and 25C.

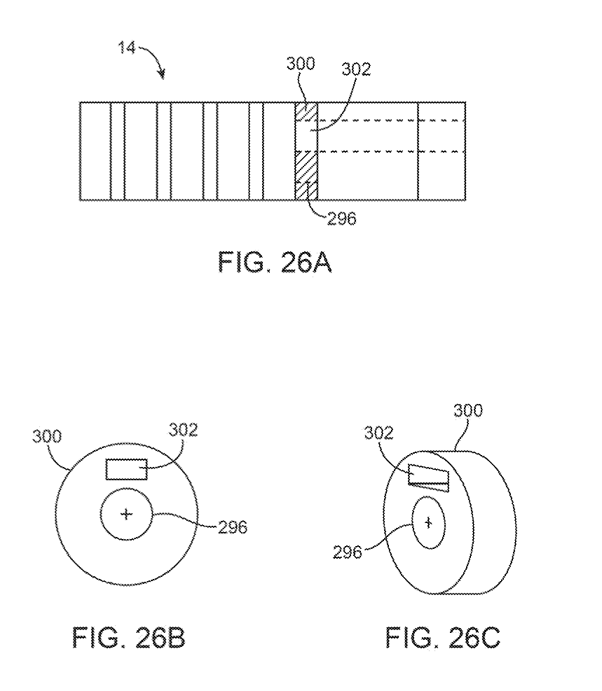

Another variation is shown in the side, end, and perspective views of FIGS. 26A to 26C which illustrates an electrode assembly 14 having an adjacently secured barrier segment 300, e.g., insulative disc, which defines a sensor opening 302 and core wire receiving channel 296. The sensor opening 302 may be configured as a passage, e.g., rectangular, which is sized to fit the pressure sensor through without necessarily contacting the pressure sensor so as to limit any transfer of stresses. The sensor opening 302 may also be scaled in size once the sensor has been placed to allow for its uninhibited passage

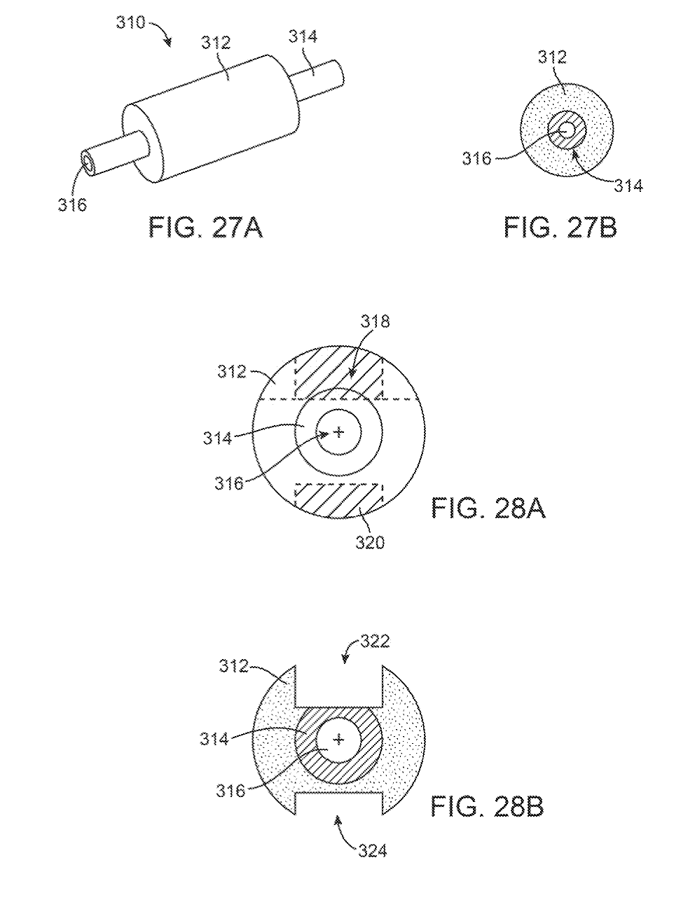

Yet another variation is illustrated in the perspective and end views of FIGS. 27A and 27B which show an electrode assembly 310 which may be formed from a conductive tube 312 having a length of, e.g., 0.050 to 0.060 in., and a diameter of, e.g., 0.007 in., fabricated from a metallic material, e.g., stainless steel, platinum-iridium, etc. The conductive tube 312 may be attached or otherwise connected over an insulative tube 314, e.g., polyimide, etc., having a diameter of, e.g., 0.005 in., which may provide structural support to the electrode assembly 310 by holding and maintaining a position of each of the conductive segments as well as providing electrical insulation. The insulative tube 314 may define a core wire channel through which the core wire may be positioned.

With the conductive tube 312, portions of the tubing may be removed to provide for space into which the pressure sensor die may be positioned. One example is shown in the end views of FIGS. 28A and 28B which illustrate how portions of the conductive tube 312 as well as portions of the insulative tube 314 may be removed as indicated by the removed section 318. The removed section 318 may have a width of, e.g., 0.007 in., and a height of, e.g., 0.0035 in., while an optionally removed section 320 may have a width of, e.g., 0.009 in., as shown in FIG. 28A. The dimensions of the removed sections 318, 320 may be varied depending upon the size of the pressure sensor die used as well as the number of conducting wires. FIG. 28B illustrates the end view of the assembly having the sections 318, 320 removed to provide for a sensor channel 322 as well as an optional channel 324, e.g., for passage of wires.

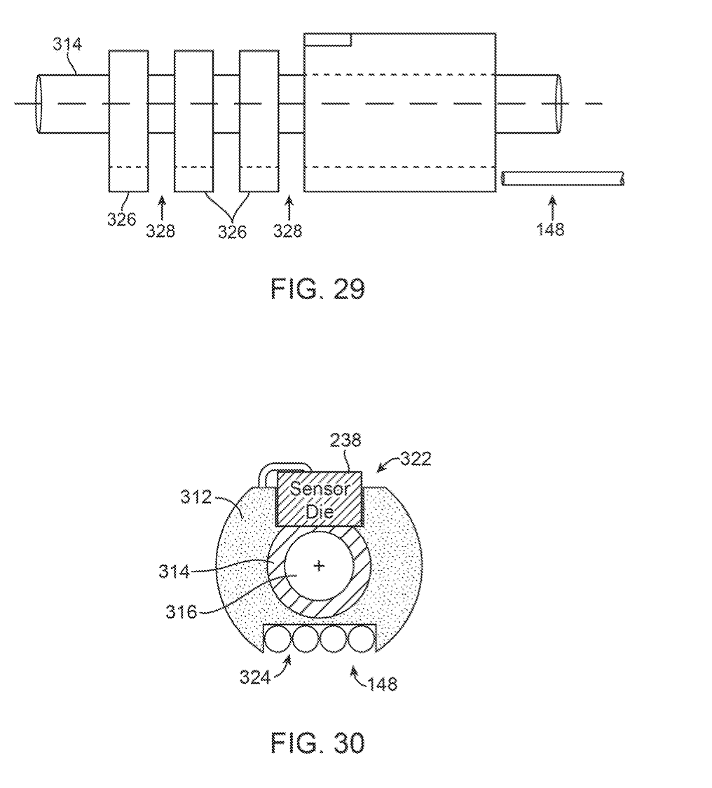

With the respective channels formed, segments may be formed by the conductive tube 312 by removing selective portions of the material. An example is shown in the side view of FIG. 29 which illustrates portions of the conductive tube 312 removed to form conductive segments 326. The formed gaps 328 between each of the conductive segments 326 where material has been removed may have a width of, e.g., 0.001 to 0.002 in., to provide for the placement of electrically insulative materials within. FIG. 30 illustrates an end view of the conductive segments 326 having a pressure sensor die 238 positioned along the sensor channel 322 and one or more conductive wires 148 positioned along the optional channel 324. It should be noted that while a method of obtaining the metal pattern on a insulative material is described, other methods such as selectively metalizing a 3D polymer surface (such as a cylinder or a rectangle with required features such as a core wire hole) via vapor deposition and photo masking it is feasible to create similar patterns and achieve the desired function.

It is intended that any of the various manufacturing and assembly processes described herein for the sensor die and/or electrode assembly may be combined in any combination as practicable. For instance, any of the assembly methods and apparatus for integrating the electrode assembly along a guidewire may be applied in combination with any of the assembly methods and apparatus for integrating the sensor along the guidewire as well. Hence, each of the variations described may be utilized alone or in any number of combinations as well.

The applications of the devices and methods discussed above are not limited but may include any number of further applications. Moreover, modification of the above-described assemblies and methods for carrying out the invention, combinations between different variations as practicable, and variations of aspects of the invention that are obvious to those of skill in the art are intended to be within the scope of the claims.

What is claimed is:

1. A method for assembling a guidewire, comprising:
providing a core wire having a tapered distal section;
securing a sensor package having one or more conductive wires to the core wire by passing the core wire through a wire receiving channel defined through or along the sensor package;
securing the one or more conductive wires to the core wire; and
encasing the one or more conductive wires and the core wire.

2. The method of claim 1 wherein securing the one or more conductive wires to the core wire comprises winding the conductive wires about the core wire.

3. The method of claim 1 wherein the sensor package defining the wire receiving channel comprises one or more electrodes separated by one or more corresponding insulative segments between each of the electrodes.

4. The method of claim 1 wherein the sensor package defining the wire receiving channel comprises a pressure sensor die mounted on a platform.

5. The method of claim 4 wherein the platform comprises conductive features spaced correspondingly to conductive pads located on the pressure sensor die.

6. The method of claim 4 wherein the pressure sensor die is attached to the platform via a flip chip bonding.

7. The method of claim 4 wherein the pressure sensor die is attached to the platform via wire bonding.

8. The method of claim 1 further comprising securing a pressure sensor die assembly along the guidewire in proximity to the sensor package having one or more electrodes.

9. The method of claim 8 further comprising electrically coupling at least one of the electrodes to the pressure sensor die assembly.

10. The method of claim 1 wherein the one or more conductive wires are insulated from each other by an insulative coating.

11. The methods of claim 10 wherein the insulated conducting wires are further coated with a metal layer.

12. The method of claim 1 wherein encasing comprises encapsulating the one or more conductive wires and core wire within a polymer.

13. A method for assembling a guidewire, comprising:
providing a distal core wire positioned within a distal coil section;
securing one or more conductive wires about a proximal core wire;
attaching a terminal end of the distal core wire to a terminal end of the proximal core wire;
positioning one or more electrodes over the proximal core wire and proximal to the distal coil section; and
electrically coupling the one or more electrodes to the one or more conductive wires.

14. The method of claim 13 wherein providing a distal core wire comprises providing the distal coil section.

15. The method of claim 13 wherein securing one or more conductive wires comprises winding the conductive wires about the proximal core wire.

16. The method of claim 13 wherein attaching a terminal end comprises welding the distal core wire to the proximal core wire.

17. The method of claim 13 wherein positioning one or more electrodes comprises positioning the one or more electrodes having one or more corresponding insulative segments between each of the electrodes.

18. The method of claim 13 further comprising positioning a proximal coil section over the proximal core wire and into contact against a proximal end of the one or more electrodes.

19. The method of claim 13 further comprising positioning a sensor die assembly along the guidewire in proximity to the one or more electrodes.

20. The method of claim 19 wherein electrically coupling comprises coupling at least one of the electrodes to the sensor die assembly.

21. A method for assembly a guidewire, comprising:
forming a narrowed distal end section of a hypotube;
attaching the narrowed distal end to a proximal end of a core wire; and,
positioning an electrode assembly about the core wire and/or narrowed distal section.

22. The method of claim 21 wherein forming a narrowed distal end section comprises forming a shoulder near a distal end of the hypotube prior to forming the narrowed distal end section.

23. The method of claim 21 wherein forming a narrowed distal end section comprises tapering the distal end section to a diameter which corresponds to a diameter of the proximal end of the core wire.

24. The method of claim 21 wherein attaching the narrowed distal end comprises welding the narrowed distal end to the proximal end of the core wire.

25. The method of claim 21 wherein positioning an electrode assembly comprises securing the electrode assembly between a distal coil section and a proximal coil section of the guidewire.

26. The method of claim 21 further comprising positioning a sensor die assembly along the guidewire in proximity to the electrode assembly.

27. A method for attaching one or more conductive wires to a surface, comprising:
positioning a sensor die within a recess of a jig;
introducing one or more conductive wires along a respective channel defined along the jig, wherein the respective channels extend from a corresponding opening along the jig and towards a conductive pad positioned along a surface of the sensor die positioned within the recess.

28. The method of claim 27 further comprising positioning an endcap adjacent to the sensor die positioned within the recess, wherein the end cap defines one or more openings therethrough corresponding to the one or more conductive wires.

29. The method of claim 28 further comprising attaching a respective terminal end of the one or more conductive wires to a corresponding conductive pad.

30. The method of claim 28 further comprising attaching the one or more conductive wires to the endcap.

31. The method of claim 28 further comprising removing the sensor die and conductive wires attached to the sensor die from the jig and securing to a guidewire.

32. The method of claim 31 further comprising integrating an electrode assembly to the guidewire.

33. A method of forming a guidewire assembly, comprising:
providing a core wire having a reduced section therealong;
positioning at least one conductive segment along the reduced section, wherein the conductive segment defines a receiving channel sized to correspond to the reduced section; and,
positioning at least one insulative segment along the reduced section adjacent to the conductive segment, wherein the insulative segment defines a receiving channel sized to correspond to the reduced section.

34. The method of claim 33 wherein the reduced section defines a keyed cross-sectional area.

35. The method of claim 33 wherein the at least one conductive segment further defines one or more openings for passage of a corresponding conductive wire.

36. The method of claim 33 wherein the at least one conductive segment further defines a channel sized for receiving a sensor die.

37. The method of claim 33 wherein the at least one insulative segment further defines a channel sized for receiving a sensor die.

38. The method of claim 33 further comprising securing a sensor die along the at least one conductive segment and at least one insulative segment.

39. The method of claim 38 further comprising securing the core wire within a distal coil section and a proximal coil section.

40. A method of forming a guidewire assembly, comprising:
providing a core wire having an insulative coating therealong;
securing a conductive tubing over a portion of the insulative coating;
removing at least one portion of the conductive tubing to form a receiving channel defined along a length of the core wire; and
removing annular portions of the conductive tubing such that segments of the conductive tubing form segments which are separated from one another along the length of the core wire.

41. The method of claim 40 wherein removing at least one portion comprises forming the receiving channel sized to receive a sensor die along the length.

42. The method of claim 40 wherein removing at least one portion further comprises removing a second portion along the length of the core wire opposite to the at least one portion.

43. The method of claim 42 further comprising positioning one or more conductive wires along the second portion.

44. The method of claim 40 wherein removing annular portions comprises removing the annular portions having a thickness of 0.001 to 0.002 in.

45. The method of claim 40 further comprising placing an insulative material between the segments of conductive tubing.

46. The method of claim 45 further comprising securing a sensor die along the segments of conductive tubing and insulative material.

47. The method of claim 40 further comprising securing the core wire within a distal coil section and a proximal coil section.
